(19) United States
(12) Patent Application Publication
(10) Pub. No.: US 2022/0202970 A1
(43) Pub. Date: Jun. 30, 2022

(54) Bio-Film Eradication

(71) Applicants: Stephen Almeida, Tampa, FL (US); James Kerr, Scarborough, ME (US)

(72) Inventors: Stephen Almeida, Tampa, FL (US); James Kerr, Scarborough, ME (US)

(21) Appl. No.: 17/212,640

(22) Filed: Mar. 25, 2021

Related U.S. Application Data
(60) Provisional application No. 62/994,669, filed on Mar. 25, 2020.

(51) Int. Cl. A61L 2/10

(52) U.S. Cl. CPC A61L 2/10; A61L 2202/10; A61L 2202/20

(57) ABSTRACT

The present application for patent relates to the field of bio-film sterilization and more specifically to the field of biofilm sterilization of fluid delivery systems which store and deliver fluids on demand.

### REFERENCE TO PRIOR FILED APPLICATIONS

This application claims the benefit of U.S. Provisional Application No. 62/994,669 filed 25 Mar. 2020 under 35 U.S.C. § 119(e); which application is incorporated by reference herein in their entirety.

### FIELD OF THE INVENTION

The present application for patent is in the field of biofilm sterilization and more specifically is in the field of biofilm sterilization of components of fluid delivery systems which store and deliver fluids on demand.

### BACKGROUND

There are a number of systems in which fluids are stored and maintained until needed, such as, for example, coffee delivery systems, soda machines and other similar devices which deliver fluids on demand from a holding reservoir and through delivery components. These systems can harness and promote growth of bacteria, viruses, molds and other infectious microorganisms within the fluid reservoir as well as any lines or conduits that deliver the fluids, especially when these systems are inactive for short periods of time, in the form of bio-films.

Biofilms are complex structures derived from microorganisms that adhere to the surfaces of storage components and delivery lines and conduits and which protect the internal cellular constituents of the film from the external environment. The external surface protective layer is made of water channels and extracellular polymeric substances (EPS). Biofilms such as Pseudomonas Aeruginosa are difficult to eradicate due to their protective architecture. Biofilms form on the insides of tubing, tanks, reservoirs and other structures which come into contact with liquids, particularly aqueous systems and other media which promote the growth of microorganisms, such as, for example, coffee makers, beverage dispensers, milk processing plants, ice cream processing plants, meat processing plants, and almost all food processing plants and processes as well as liquid processing/dispensing devices causing health risks for the consumer. The biofilm's EPS protective layer protects the biofilms from the onslaught of enzymes, chemical cleaners, ionic insults, and other forms of removal making eradication exceedingly difficult. Thus, there is a continuing need for new and better processes and equipment that help to reduce or eradicate the efficacy of bio-films and their component microorganisms.

### BRIEF DESCRIPTION OF THE DRAWINGS

FIG. 1 depicts one embodiment of the current invention.

FIG. 2 depicts another embodiment of the current invention.

FIG. 3 depicts a further embodiment showing 2 UVC emitting devices working simultaneously.

### BRIEF DESCRIPTION OF THE DISCLOSURE

As used herein, the conjunction “and” is intended to be inclusive and the conjunction “or” is not intended to be exclusive unless otherwise indicated. For example, the phrase “or, alternatively” is intended to be exclusive.

As used herein the phrase “at least one” means one or more as desired.

As used herein the term “transparent” means the passage of at least 50% of electromagnetic radiation.

As used herein the term “significantly” means more than 50%.

In a first embodiment, disclosed and claimed herein are fluid sterilizing devices comprising UVC transparent materials, and at least one UVC radiation emitting component arranged to impinge on the outer surfaces of the UVC transparent materials.

In a second embodiment, disclosed and claimed herein are fluid sterilizing devices of the above embodiment wherein the UVC transparent materials comprise quartz, fluorinated polymers, or fluorinated ethylene propylene polymer or combinations thereof.

In a third embodiment, disclosed and claimed herein are fluid sterilizing devices of the above embodiments wherein the UVC transparent materials are used to construct fluid storage devices and delivery systems.

In a fourth embodiment, disclosed and claimed herein are fluid sterilization devices of the above embodiments wherein the delivery systems comprise tubing, conduits, pipes, flow control mechanisms, capturing vessels, hoses, valves, and processing vessels.

In a fifth embodiment, disclosed and claimed herein are fluid sterilization devices of the above embodiments comprising more than one UVC emitting component wherein the UVC emitting devices are arranged facing each other on opposite sides of the fluid storage device or fluid delivery systems wherein a bio-film receives radiation from both the underside and the top side of the bio-film.

### DETAILED DESCRIPTION OF THE DISCLOSURE

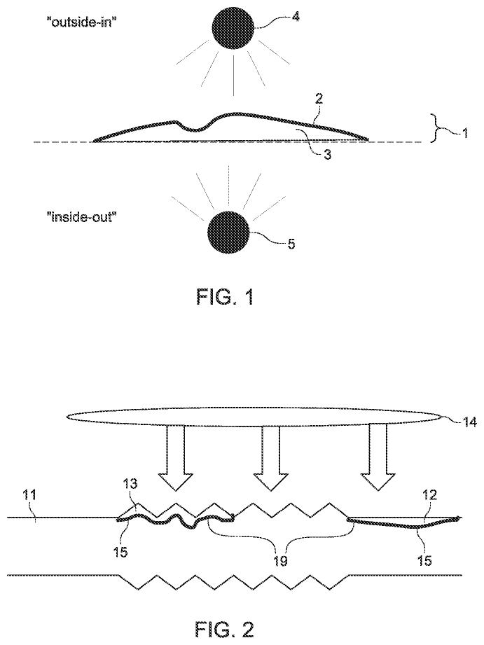

Studies have shown that exposing the underside of a biofilm (“Inside-out” 4 in FIG. 1) to UVC as opposed to exposing the outer EPS layer of the biofilm (“outside-in” 5 in FIG. 1), requires 47% less UVC dosage due to the fact that there is no EPS layer to protect the internal cellular constituents. This current disclosure takes advantage of this physical characteristic to improve the eradication or deactivation of biofilm by illuminating the underside of the biofilm which does not have a protective EPS layer in fluid delivery devices. The current disclosure presents a solution tom this difficult problem.

The current disclosure describes a fluid sterilizing device comprising UVC transparent materials, and at least one UVC radiation emitting component arranged to impinge on the outer surfaces of the UVC transparent materials. The devices of the current disclosure are constructed of materials that are transparent to UVC sterilizing radiation. Such materials include, for example, quartz, fluorinated polymer and polymer blends, fluorinated ethylene-propylene copolymer and the like. Any material known in the art can be used to construct the devices of the current disclosure. The devices comprise at least one UVC emitting components arranged so that the sanitizing radiation impinged on the materials constructed of UVC transparent materials, so that, for example, the inside of the construction receives enough radiation through the UVC transparent materials so as to deactivate or eradicate any bio-film or microorganism contaminant as shown in FIG. 1. FIG. 1 shows outside-in exposure of the UVC lamp 4 onto a bio- film 1, which is composed of a protective EPS layer 2 and internal cellular constituents, 3. The Figure also shows the exposure of bi-film 1 from the inside-out by a UVC lamp 5 positioned to expose the bio-film from the underside.

FIG. 2 illustrates an embodiment of the current disclosure. Liquid delivery system 11, which includes smooth areas as well as corrugated or non-smooth surface has bio-films 19 in both places. UVC lamp 14 exposes the bio-films from the underside walls of the delivery system 11 exposing the internal cellular constituents 12 and 13 which bypasses the protective EPS layer 15, thus providing an increase in sterilization efficiency and effectiveness.

More than one UVC emitting component may be present and distributed throughout the device as require or desired.

The fluid storage and delivery systems which can benefit from the current disclosure include, for example, tubing, conduits, pipes, flow control mechanisms, capturing vessels, hoses, valves, and processing vessels. More specific examples of fluid storage and delivery systems include, for example, coffee makers, beverage dispensers, milk processing plants, ice cream processing plants, meat processing plants, all dairy production facilities and almost all food processing plants and processes as well as liquid, or paste, processing/dispensing devices can be susceptible to harmful microorganisms potentially cause health risks for the consumer.

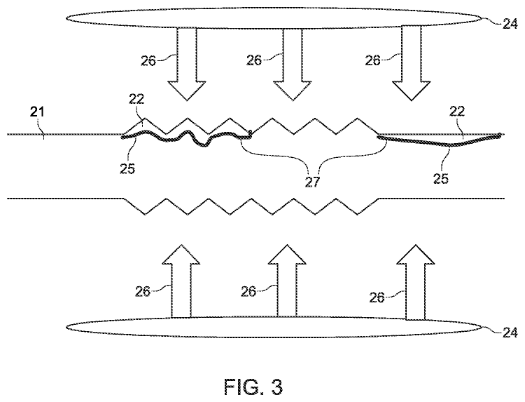

In another embodiment of the current disclosure, fluid sterilizing devices are described comprising UVC transparent materials, and more than one UVC radiation emitting component wherein one UVC emitting component is positioned to impinge on the outer surface of the fluid storage or delivery system constructed of UVC transparent material and a second UVC emitting component is positioned to impinge on the outer surface of the other side of the fluid storage or delivery system, wherein the two UVC emitting components are directly facing each other. In this manner any bi-film present on the fluid storage or delivery system can benefit from both a highly effective sterilizing exposure and a top-side exposure which is less than efficient as underside exposure but adds to the overall total UVC sterilizing energy. FIG. 3 illustrates one dual UVC exposure embodiment. UVC lamp 24a is positioned on one side of the outer surface of a fluid delivery system 21. On the other side of the of the fluid delivery system and positioned to be facing 24a is UVC lamp 24b. The UVC emission components can work simultaneously to expose biofilms 27 with UVC emissions. Biofilms 27 are composed of internal cellular components 22 and an outer protective EPS layer 25. In operation lamp 24a exposes 26 the bio-films from the underside while lamp 24b expose 6 the bio-films from the top side.

We claim:

1) A fluid sterilizing device comprising:
a. UVC transparent materials, and
b. At least one UVC radiation emitting component arranged to impinge on the outer surfaces of the UVC transparent materials and eradicate or significantly reduce

2) The fluid sterilizing device of claim 1 wherein the UVC transparent materials comprise quartz, fluorinated polymers, or fluorinated ethylene propylene polymer or combinations thereof.

3) The fluid sterilizing device of claim 2 wherein the UVC transparent materials are used to construct fluid storage devices and delivery systems.

4) The fluid sterilization device of claim 3 wherein the delivery systems comprise tubing, conduits, pipes, flow control mechanisms, capturing vessels, hoses, valves, and processing vessels.

5) The fluid sterilization devices of claim 1 comprising more than one UVC emitting component wherein the UVC emitting devices are arranged facing each other on opposite sides of the fluid storage device or fluid delivery systems wherein a bio-film receives radiation from both the underside and the top side of the bio-film.
